(19) United States
(12) Patent Application Publication
(10) Pub. No.: US 2007/0142811 A1
(43) Pub. Date: Jun. 21, 2007

(54) Absorbent products having complementary fragrances and a package for storing absorbent products

(76) Inventor: Brooke Elizabeth Lais, Cincinnati, OH (US)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(21) Appl. No.: 11/449,100

(22) Filed: Jun. 8, 2006

Related U.S. Application Data
(60) Provisional application No. 60/751,011, filed on Dec. 16, 2005.

(51) Int. Cl. A61F 13/15

(52) U.S. Cl. 604/385.06; 604/385.02

(57) ABSTRACT

A package for storing and dispensing absorbent articles and wipe articles. The package has one or more walls defining an interior storage space and a means for accessing the interior storage space. The package comprises one or more first articles disposed in the interior storage space wherein at least one of the one or more first articles is an absorbent article. The package further comprises one or more second articles disposed in the interior storage space wherein at least one of the one or more second articles is an enclosed pouch having a wipe article disposed therein. At least one of the one or more second articles is joined to at least one of the one or more first articles. The package still further comprises one or more sensory cues on the package, wherein the one or more sensory cues on the package relate to indicia on the one or more second articles. The one or more sensory cues on the package also relate to the wipe article.

### CROSS REFERENCE TO RELATED APPLICATION

This application claims the benefit of U.S. Provisional Application No. 60/751,011, filed Dec. 16, 2005.

### FIELD OF THE INVENTION

The present invention relates generally to the field of disposable absorbent articles and wipes and packages therefor.

### BACKGROUND OF THE INVENTION

Wearable absorbent articles for absorbing and handling body exudates, such as diapers, sanitary napkins, panty liners, adult incontinence pads and the like are widely known in the art. Additionally, some absorbent articles may contain a fragrance producing composition to mask smells related to body exudates contained on or in the absorbent article as well as to provide aromatherapy for a person. Due to different personal hygienic needs articles like sanitary napkins are often individually wrapped, whereas diapers, panty liners or adult incontinence pads may be packaged as a stack in a common package. It has also been recognized that changing such absorbent articles can be a hygienic challenge, as portions of the user's body often need to be cleaned, and the hands of the person changing the article may become contaminated. This can be perceived as unhygienic.

It is also widely known in the art to use wipes for cleaning skin portions soiled with body exudates. For example, the use of moistened baby wipes is widely practiced. Such wipes are made of a nonwoven material and oftentimes include a lotion or aqueous solution to ease cleaning and to deliver skin benefits to the skin portion treated therewith. Additionally the wipes may be scented in order to mask the smells associated with the cleaning process and/or to provide a pleasant scent to the user. For maintaining the moistness and the fragrance of the wipe these wipes are packaged in vapor impermeable packages. Typically, cleaning wipes are packaged in stacks in a common package, such as in the case of moist baby wipes. In other instances, such as in case of cleaning wipes supplied in airplanes, there is only one wipe individually packaged in a wrapper. Importantly the scent contained on the wipe may conflict with any scent contained on another personal care article such as, for example, a sanitary napkin resulting in an undesirable experience for the user.

Currently, when attempting to change a soiled absorbent article, such as a soiled sanitary napkin, the user or the person changing the article has to carry the package containing the article for changing as well as another package containing the wipe for cleaning. This is clearly disadvantageous because opening, handling, and disposing of numerous packages containing a multiplicity of items is required.

One solution to the above problem is to provide an individual absorbent article comprising an individually-packaged moistened wipe in or on the package of the absorbent article. In other words, the absorbent article and wet wipe are fixedly combined with each other. These combined absorbent articles are an improvement over prior approaches because the number of parts and also waste parts, which must be handled, is reduced as the packages of the absorbent article and the wipe are affixed to each other. However, one disadvantage of this approach is that a person may not recognize that the sanitary napkin and wipe are being marketed together as a single item without the proper kind of packaging, and thus the person will not be aware of a possible solution to the disadvantage discussed above.

Yet another disadvantage associated with the packaging and marketing of an absorbent article and wipe combination is that even with proper packaging a person who is in a hurry or simply does not wish to inconvenience herself by taking the time to stop and examine a package in order to determine the contents within may not realize that the package contains a wipe and absorbent article combination. Not only may a person not realize the foregoing, but she also may not know whether the wipe is a wet wipe or a dry wipe. Many users of consumer goods prefer one type or brand of good to another, and thus another disadvantage of previous attempts at marketing an absorbent article and wipe combination is that without some indication or sensory cue on the package a person may be unaware that the absorbent article and the wipe are the same products that she might normally purchase as separate items.

Oftentimes a user of an absorbent article may not be at home when the need arises to use the absorbent article. As a result the person may need to find an away from home place in order to address whatever need she may have. One such place might be a public restroom. While a public restroom may provide a place in which to take care of personal sanitary needs, often it does not provide the lighting, space or shelving to conveniently use absorbent articles and clean oneself with a wipe if needed. While having a wipe attached to the absorbent article alleviates some of the this inconvenience another problem may arise when there is no easily identifiable means on the wipe to indicate the proper place to grasp the package containing the wipe in order to open it or if the joining means affixing the wipe package to the absorbent article has insufficient strength to sustainably hold the wipe to the absorbent article.

Accordingly it would be desirable to provide a scented absorbent article with a scented wipe attached thereto such that the absorbent article and the wipe have related, complementary, synergistic or identical fragrances. It would also be desirable to provide an absorbent article with a wipe attached thereto contained in a package that allows a person to see the wipe attached to the absorbent article inside the package. It would further be desirable to provide an absorbent article with a wipe attached thereto contained in a package that has one or more sensory cues to communicate to a person that there is an absorbent article with a wipe attached thereto contained within the package, that the absorbent article and the wipe are the same products that are marketed individually, and/or that the wipe is a wet wipe. Finally, it would still further be desirable to provide an absorbent article with a wipe attached thereto where the joining means affixing the wipe to the absorbent article has sufficient strength to hold the absorbent article and the wipe together while the wipe is being opened, and to have one or more sensory cues on the package containing the wipe to communicate to a person where to grasp the wipe package in order to open it.

### SUMMARY OF THE INVENTION

In order to provide a solution to the inconveniences set forth above in relation to the prior art, the invention provides a novel absorbent product combination and package therefore.

In one aspect of the present invention there is provided a package for storing and dispensing absorbent articles and wipe articles. The package has one or more walls defining an interior storage space and a means for accessing the interior storage space. The package comprises one or more first articles disposed in the interior storage space wherein at least one of the one or more first articles is an absorbent article. The package further comprises one or more second articles disposed in the interior storage space wherein at least one of the one or more second articles is an enclosed pouch having a wipe article disposed therein. At least one of the one or more second articles is joined to at least one of the one or more first articles. The package still further comprises one or more sensory cues on the package, wherein the one or more sensory cues on the package relate to indicia on the one or more second articles. The one or more sensory cues on the package also relate to the wipe article.

### BRIEF DESCRIPTION OF THE DRAWINGS

FIG. 1 is a cross-sectional side view of one embodiment of the present invention.

FIG. 2 is a cross-sectional side view of an alternative embodiment of the present invention.

FIG. 3 is a plan view of one embodiment of the present invention.

FIG. 4 is a plan view of one embodiment of the present invention.

FIG. 5 is a plan view of one embodiment of the present invention.

FIG. 6 is a plan view of one embodiment of the present invention.

FIG. 7 is an isometric view of an alternative embodiment of the present invention.

FIG. 8 is an isometric view of an alternative embodiment of the present invention.

FIG. 9 is an isometric view of an alternative embodiment of the present invention.

FIG. 10 is a plan view of an alternative embodiment of the present invention.

FIG. 11 is a cross-sectional side view of an alternative embodiment of the present invention.

### DETAILED DESCRIPTION OF THE INVENTION

FIG. 1 shows a cross-sectional side view of one embodiment of the present invention. The absorbent product combination 21 shown comprises a sanitary napkin 1 and an enclosed pouch 3 joined to the sanitary napkin 1 by a joining means 2. As used herein the term “joined” refers to the condition where a first member or component is attached, affixed, or otherwise physically connected to a second member or component either directly or indirectly. An example of an indirectly joined member or component might be where the first member or component is affixed, or connected to an intermediate member or component which in turn is affixed or connected to the second member or component. The joined relationship between the first member or component and the second member or component may be permanent or temporary. As used herein the term “permanently joined” typically refers to a connection that cannot be unattached without at least partially destroying one of the attached components. As used herein the term “temporarily joined” typically refers to a connection that is meant to be easily releasable such as, for example, a connection that enables the pouch to be releasably joined to the sanitary napkin. One example of a suitable joining means 2 might be an adhesive, and while it may be desirable to use an adhesive, such as a pressure sensitive adhesive, to join the enclosed pouch 3 to the absorbent article 1 it is to be understood that any suitable means for joining the enclosed pouch 3 to the absorbent article 1 discussed herein or known to those having ordinary skill in the art is contemplated by the present invention.

While FIG. 1 shows a sanitary napkin it is to be understood that the present invention is not limited to sanitary napkins, but contemplates any wearable absorbent article such as, for example, disposable diapers, adult incontinence pads, panty liners, and the like. It is to be further understood that while FIG. 1 shows a sanitary napkin with no releasable wrapper the present invention contemplates embodiments having sanitary napkins that may include a releasable wrapper that is either joined to or not joined to the sanitary napkin and where the releasable wrapper may either partially or completely surround the sanitary napkin.

The sanitary napkin 1 shown in FIG. 1 has a body-facing side or face 7 and an opposed garment-facing side or face 8. The enclosed pouch 3 has an outwardly facing side 22 and an opposed inwardly facing side 23. The inwardly facing side 23 of the enclosed pouch can be joined to the garment facing side 8 of the sanitary napkin 1. The sanitary napkin 1 can be used to collect vaginal discharges such as, for example, menses, and to prevent soiling of the wearer's clothing by such discharges. A particularly suitable sanitary napkin may be made in accordance with U.S. Pat. No. 5,569,230, issued Oct. 29, 1996 to Fisher et al. The sanitary napkin 1 can also include one or more fragrance compositions disposed together or separately on at least a portion of the sanitary napkin 1.

The enclosed pouch 3 joined to the sanitary napkin 1 can be made such that the enclosed pouch 3 defines an internal storage space 15. The enclosed pouch 3 may also have a wipe article 4 disposed within the internal storage space 15. The enclosed pouch 3 can also be vapor impermeable. Further, the enclosed pouch 3 can be capable of being flushed in a toilet (i.e., it is “flushable”), and disintegrates sufficiently when flushed in a toilet and when being transported in the sewer system so it does not plug any element of a sewer system. Materials that meet such criteria include, but are not limited to, silicone-treated polyvinyl alcohol films, or films coated with a polyvinyl alcohol, tissue coated or impregnated with polyvinyl alcohol, or similar or other water soluble materials. The enclosed pouch 3 may further comprise a unitary construction, however, it is to be understood that embodiments where the enclosed pouch 3 is not of unitary construction are also contemplated by the present invention. The term unitary construction as used herein refers to an enclosed pouch 3 that has been made by a process utilizing a single undivided piece of starting material wherein the starting material is folded about one axis to form one edge of the enclosed pouch 3, and then sealed on the remaining edges to define an enclosed interior storage space 15.

For enclosing wet wipes, i.e. wipe articles at least partially saturated with a liquid solution, it may be advantageous to have seals around all sides of the enclosed pouch 3 that can prevent substantial evaporation of the liquid solution from the time the product is manufactured to the time it is used by the consumer. A more detailed description of wet wipes and the liquid solutions contained thereon is given below. Any number of sealing mechanisms may be used including, but not limited to, heat and/or pressure seals, ultrasonic seals, glue seals, zippered track sealing systems such as Dow Company's “ZIP LOCKS” or any combination thereof and the like. In addition, the enclosed pouch 3 may be lined with foil, or some other suitable material to further reduce evaporation. The enclosed pouch 3 can comprise extremely thin and flexible materials known to those having ordinary skill in the art in order to facilitate storage of the absorbent product combination 21 in a variety of portable storage locations.

As shown in FIG. 1 the wipe article 4 can be disposed within the interior storage space 15 defined by the enclosed pouch 3. The wipe article 4 may comprise a nonwoven fabric. The nonwoven fabric may further comprise synthetic fibers such as, for example, rayon, polyethylene, polypropylene, or polyester or natural fibers such as, for example, cellulose or cotton. The wipe article 4 can be made of a nonwoven material or a wet-laid material in a manner similar to toilet tissue or facial tissue. The wipe article 4 can be capable of being flushed in a toilet (i.e., it is “flushable”), such that it disintegrates sufficiently when flushed in a toilet and when being transported in the sewer system so it does not plug any element of a sewer system. One material that may be suitable for use as a wipe article 4, and as an enclosed pouch 3 is the hydro-entangled fabric described in U.S. Pat. No. 4,755,421, issued to Manning, et al. on Jul. 5, 1988.

The size of the wipe article 4 can vary. The wipe article 4 can be greater than or equal to about 4 square inches (about 25 square centimeters) in size, greater than or equal to about 9 square inches (about 50 square centimeters) in size, less than or equal to about 225 square inches (about 1,450 square centimeters) in size, between about 16 square inches (about 100 square centimeters) and about 50 square inches (about 320 square centimeters), or about 35 square inches (about 225 square centimeters) in size.

The wipe article 4 can be a cleansing wipe. The wipe article 4 can also be a hygienic cleansing wipe that may be used by the wearer to clean menses and/or other body exudates from her body. The cleaning of menses can be particularly important because when menses leaves the wearer's body, it may tend to smear over the pudendal region of the wearer's body and be retained on the wearer's skin and pubic hair. Furthermore, the menses may then dry on the skin and in the pubic hair, and make later cleansing difficult.

The inclusion of a wipe article 4 such as, for example, a hygienic cleansing wipe, with the individual sanitary napkin 1 provides several advantages. The wipe article 4 provides for physical cleansing that can increase physical comfort as well as psychological comfort by providing a feeling of cleanliness to a user. The wipe article 4 may also be used to reduce soiling of the user's panties. One mechanism that causes panty soiling is the transfer of menses from soiled body surfaces to the wearer's panties. In addition, the use of the wipe article 4 may also provide a reduction in the odor associated with menstruation.

The wipe article 4 can be a wet wipe (that is, liquid-containing) that is provided with a liquid-based cleansing solution. The wipe article 4 may also be scented. The wipe article 4 can comprise a nonwoven fabric impregnated with the liquid-based cleansing solution. The wipe article 4 can also contain substances such as silicones that tend to inhibit menses from adhering to the wearer's skin and pubic hair. The liquid-based cleansing solution can include surfactants, alcohols, fragrance compositions, antimicrobial agents, and pH buffers, or combinations thereof and the like. The liquid-based cleansing solution can also contain skin conditioning substances similar to those used in hand lotions, or any other substances known in the art for inclusion in cleansing wipes.

Other embodiments can include dry wipes or wipes containing non-aqueous cleaning solutions such as, for example, mineral oils, and the emollient described in U.S. Pat. No. 4,481,243, issued to Allen on Nov. 6, 1984.

One or more fragrance compositions can be included in the cleansing solution in order to produce a scented wipe article. However, the one or more fragrance compositions can also be included in the wipe article 4 separately from any cleansing solution that may be present such as, for example, by applying the fragrance composition to the wipe article 4 prior to or after any cleansing solution may be added. Another example might be a wipe article 4 that includes a separate component such as, for example, a carrier material comprising a fragrance composition. Additionally, the one or more fragrance compositions may be disposed together or separately on at least a portion of the wipe article 4.

While a scented wipe article may provide a desirable product to a user, it may be even more desirable to have the one or more fragrance compositions included in the wipe article 4 produce one or more fragrances that are related, complementary, synergistic or identical to one or more fragrances produced by one or more fragrance compositions contained on another personal care article such as, for example, a sanitary napkin. As used herein the term “related” means to have characteristics that can logically show or establish a connection between two or more elements. By way of example and not limitation, the fragrance of a rose and the fragrance of an orchid can be related since both fragrances share the common theme of flowers. Another example of related fragrances might include the fragrance of rain and the fragrance of flowers since both fragrances share the common theme of Spring. As used herein the term “complementary” refers to two or more elements that when detected together by a user can produce a desirable reaction in the user. Examples of desirable reactions might include, for example, feelings of enjoyment or thoughts of cleanliness. One example of complimentary elements might include the fragrances of a flower and a fruit. As used herein the term “synergistic” refers to elements that when detected together by a user produce a reaction in the user that is stronger or more desirable than the reaction created when the user detects the elements individually. For example, the fragrance of a flower, such as a carnation, and the fragrance of a spice, such as cinnamon, when detected together by a user may produce a reaction such as, for example, a feeling of enjoyment, that is stronger or more desirable than when the fragrances are detected individually. Another example of synergistic fragrances might include the fragrance produced by commercially available potpourri as compared to the individual scents comprising the potpourri. As used herein with regard to fragrances the term “identical” refers to fragrances that have identical compositions, or even if the compositions differ, they have no readily discernible differences when detected by a typical user. For example, two fragrance compositions that both produce the fragrance of a rose would be identical.

The above examples for the terms related, complementary, synergistic, and identical all involve the use of fragrance(s), however it is to be understood that other elements of the present invention may be also be related, complementary, synergistic or identical such as, for example, indicia on the package(s).

The absorbent product combination 21 may be used by a consumer in a variety of away from home environments such as, for example, a public restroom. Typically an away from home environment is not the preferred environment for using the absorbent product combination 21 due to lack of, for example, space, lighting, storage or disposal space. The result of this oftentimes necessary inconvenience is that the absorbent product combination 21 may be carried by the consumer in a variety of portable storage locations such as, for example, a pocket, a purse, a handbag, or some other portable storage location that usually accompanies the consumer on an away from home trip. Due to the wide variety of portable storage locations that may be utilized by a consumer it may be desirable to have the adhesive force of the joining means 2 be at least as great as the weight of absorbent product combination 21 in order to ensure the enclosed pouch 3 does not separate from the sanitary napkin 1 upon removal of the absorbent product combination 21 from a portable storage location.

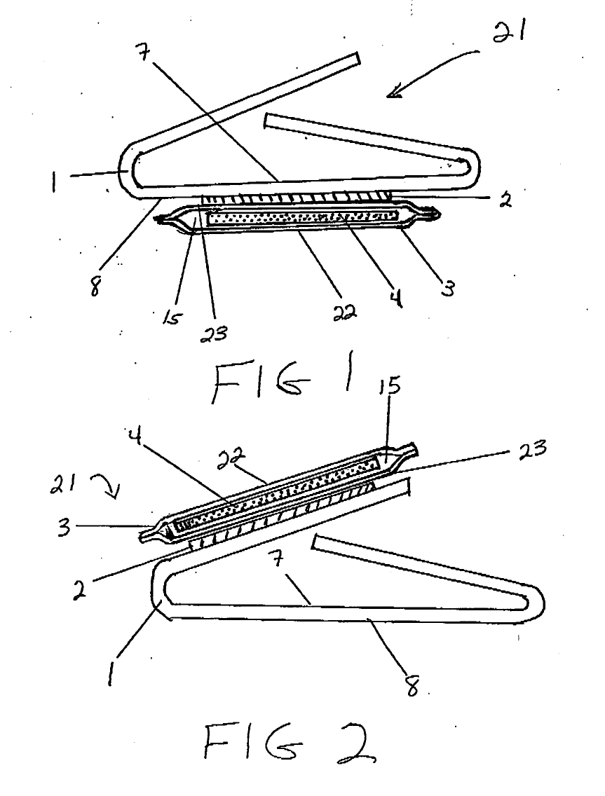

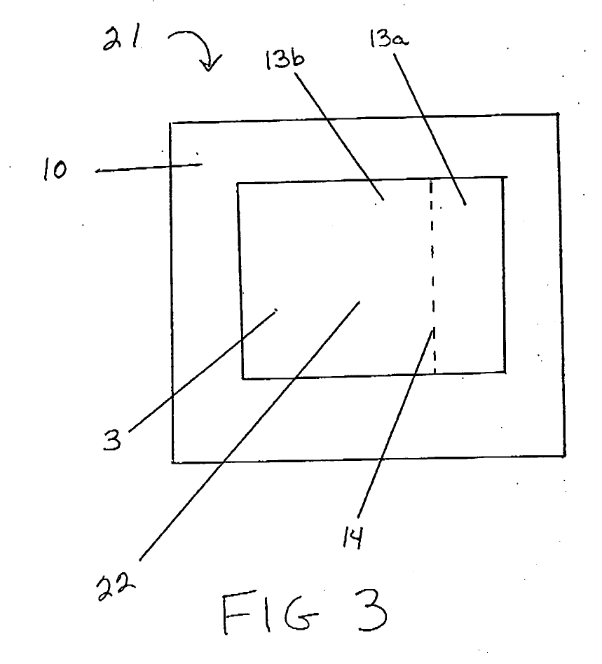

FIG. 2 shows another embodiment of the present invention. In this example the enclosed pouch 3 is joined to the absorbent article 1 at one end, as opposed to the middle as exemplified by FIG. 1. It is to be understood that the locations shown are by way of example only and that the present invention contemplates embodiments where the enclosed pouch 3 is attached to any suitable location on the sanitary napkin 1.

The enclosed pouch 3 may include a means for unsealing (or opening) the enclosed pouch 3 in order to access a wipe article 4 contained therein. FIG. 3 shows a plan view of the absorbent product combination 21 comprising an absorbent article 10 having an enclosed package 3 joined thereto. The enclosed pouch 3 has indicia 14 that define a first grasping portion 13a and a second grasping portion 13b. The first and second grasping portions 13a, 13b can comprise areas of the pouch where the user may grasp in order to facilitate opening of the enclosed pouch 3. The first grasping portion 13a can comprise about 1%-50% of the total surface area of the enclosed pouch 3, but it is to be understood that embodiments where the first grasping portion 13a can comprise a range of less than 1% to about 95% of the surface area of the enclosed pouch 3 are also contemplated by the present invention.

By applying adequate force to the first grasping portion 13a, the first grasping portion 13a may be at least partially removed thereby opening the enclosed pouch 3. The amount of force required to open the enclosed pouch 3 may be less than the adhesive force of the joining means 2 (shown in FIG. 1) thereby permitting opening of the enclosed pouch 3 by utilizing only the first grasping portion 13a without fear of removing the enclosed pouch 3 from the absorbent article 10. The peak force required to open the enclosed pouch 3 can be anywhere from about 0.5 N to about 15 N. The peak force required to open the enclosed pouch 3 can also be anywhere from about 1 N to about 10 N. The peak force required to open the enclosed pouch 3 can also be about 6 N. The peak force required to open the enclosed pouch 3 may also be greater than or equal to the adhesive force of the joining means 2 in order to permit relatively easy removal of the enclosed pouch 3 from the absorbent article 10 as desired.

One example of an opening means may include an enclosed pouch 3 with a scored or die cut line of weakness on the outwardly facing side 22, the inwardly facing side 23, or both sides. This line of weakness may allow the enclosed pouch 3 to be opened easily with a tab, easy peel label or any other conventional opening mechanism known to those having ordinary skill in the art. The line of weakness may be disposed in the same location as the indicia 14 defining the first and second grasping portions 13a, 13b, however, it is to be understood that embodiments where the opening means is disposed on any other portion of the enclosed pouch 3 are also contemplated by the present invention. Another example to facilitate easy opening of the enclosed pouch 3 might include a “Dry-edge” sticker mechanism. Yet another example of an opening means might include the enclosed package 3 that is made of a material that is relatively easy to tear such as, for example, and easy to tear film. A package comprising a material that is relatively easy to tear would have the advantage of not requiring additional processing steps such as, for example, the steps required to add a weakened portion or a perforated portion. Still other examples of suitable opening means may include, but are not limited to, releasable adhesives, releasable heat and/or pressure seals, perforations, rupture seals, tear seals, or any combination of the foregoing and the like.

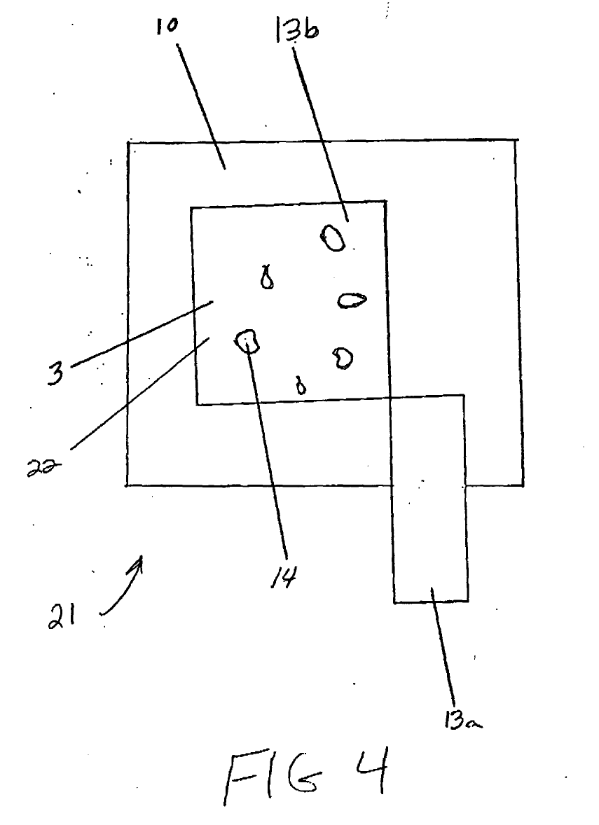

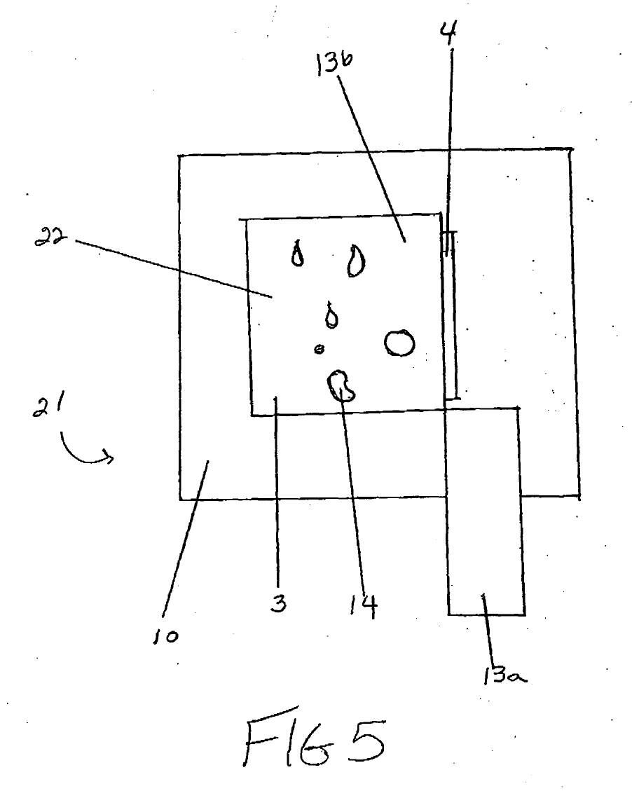

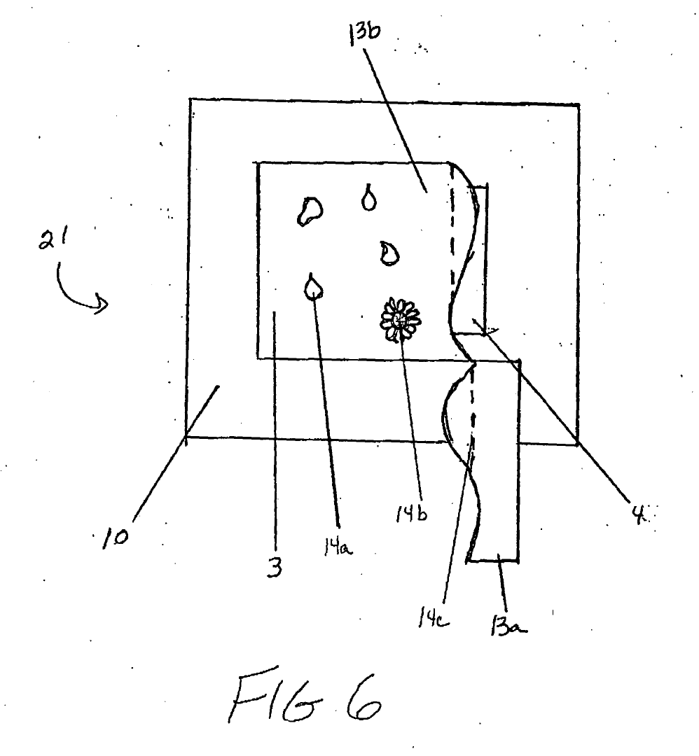

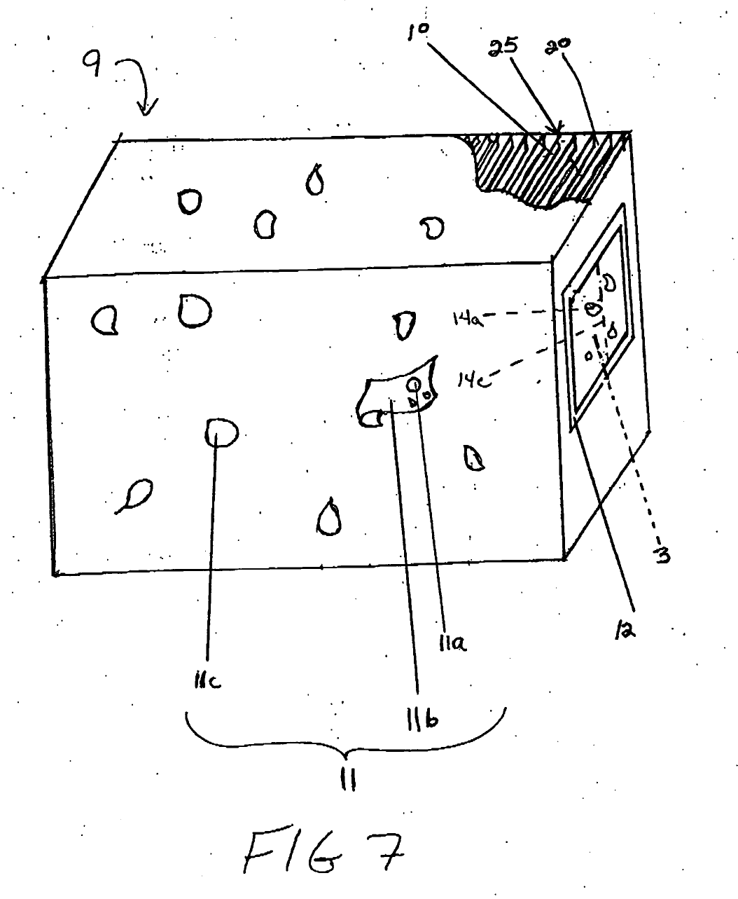

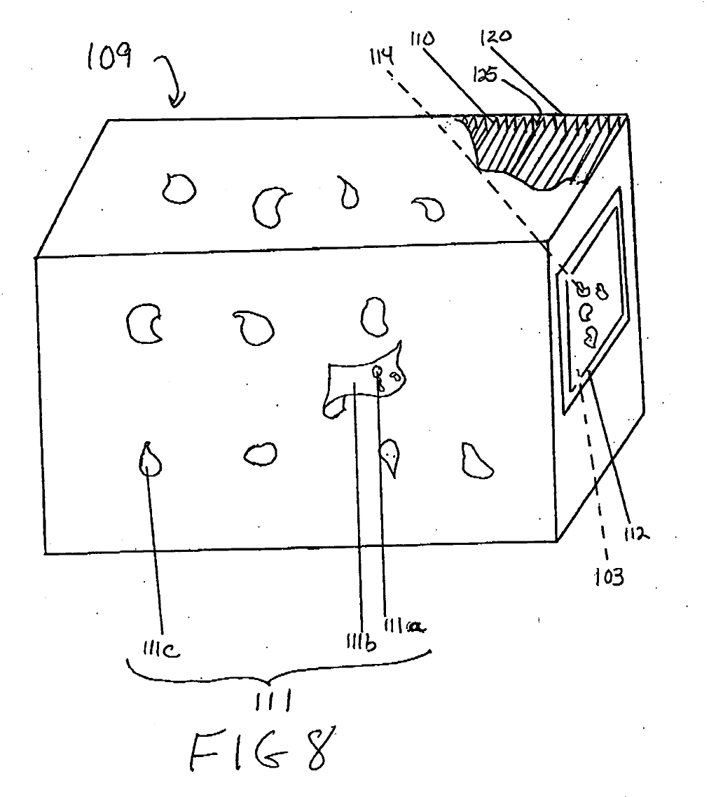

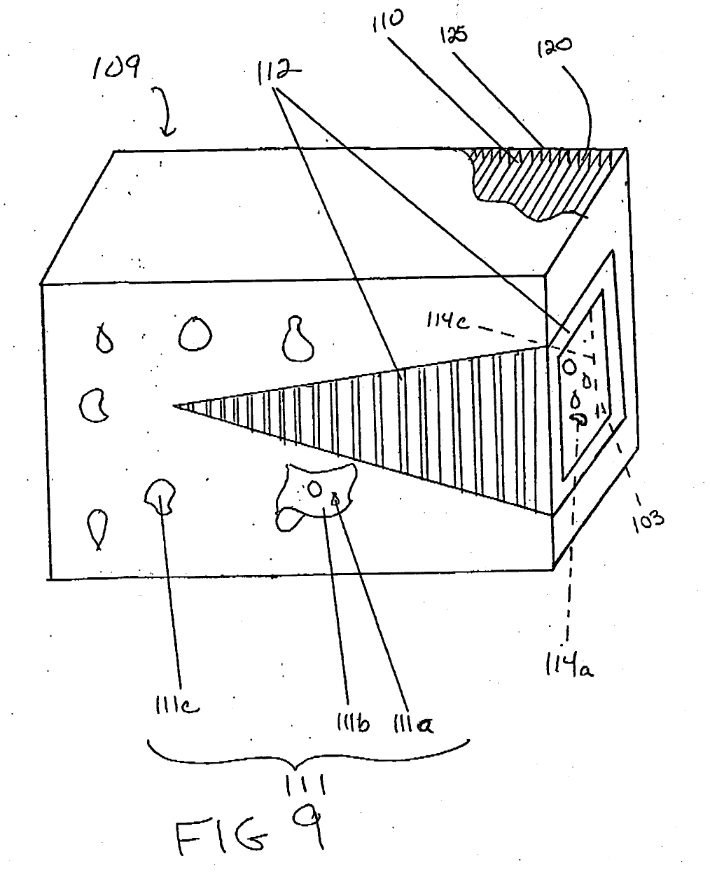

When opened, the enclosed pouch 3 may provide an opening with a generally straight edge as shown in FIG. 4, or a curved edge as shown in FIG. 6.

As previously stated the indicia 14 included on the enclosed pouch 3 can communicate to a user the areas on the enclosed pouch 3 containing the first and second grasping portions 13a, 13b. As shown in FIG. 3 the absorbent product combination 21 can include indicia 14 disposed on the outward faceable side 22 of the enclosed pouch 3. The indicia 14 in FIG. 3 include a dashed line, but it should be understood that the indicia 14 of the present invention are not limited to any particular object or shape, but may include a single color, combinations of colors, raised portions, object(s), partial objects, or any combination(s) of objects, partial objects, or any of the aforementioned and the like that may or may not form a readily discernible picture or pattern. Further, the indicia 14 may be positioned on any portion or location of the pouch 3 including but not limited to any material that may be joined to or surround the pouch 3, such that the indicia 14 are detectable by the human eye when the absorbent product combination 21 is displayed in a package as described more fully hereinbelow.

FIG. 4 shows one embodiment of the present invention wherein the enclosed pouch 3 is opened (i.e. the first grasping portion 13a is partially removed) and wherein the absorbent product combination 21 can include indicia 14 depicting water droplets disposed on the enclosed pouch 3. The indicia 14 are can be disposed on the outwardly facing side 22 of the enclosed pouch 3 in the portion of the enclosed pouch 3 containing the second grasping means 13b. In this manner the first grasping portion 13a can be recognized by a user as the portion not having indicia 14 and the second grasping portion 13b can be recognized as that portion having indicia 14.

FIG. 5 shows another embodiment of the present invention wherein an absorbent product combination 21 can comprise an enclosed pouch 3 having a wipe 4 disposed therein such that the wipe 4 is not positioned entirely within the area containing the second grasping portion 13b, and thus can be visible when the enclosed pouch 3 is opened.

FIG. 6 shows yet another embodiment of the present invention wherein the enclosed pouch 3 can include indicia depicting water droplets 14a, a flower 14b and a dashed line 14c. It may be desirable to include the indicia 14 on the enclosed pouch 3 such that they can indicate to a user not only the portions of the enclosed pouch 3 comprising the first grasping portion 13a and the second grasping portion 13b, but can also relate to the wipe article 4 disposed within the enclosed pouch 3. For instance, in the present example the depiction of a water droplet 14a can communicate to a user that the wipe article 4 contained within the enclosed pouch 3 is a wet wipe, and the depiction of a flower 14b can communicate to a user that the wipe article 4 includes the scent of a flower. The dashed line 14c can define the portions of the enclosed pouch 3 defining the first and second grasping portions 13a, 13b.

The marketing of an absorbent article with a cleansing wipe in a common package may be an efficient and desirable way of combining the utility of both types of personal care articles, and may thereby minimize any unpleasantness associated with using a personal care article. However, without an effective means to communicate to a consumer that the items are packaged together the consumer may not be aware of the advantageous product available to her.

FIG. 7 shows a package 9 having one or more walls that define an interior storage space 20 for storing one or more absorbent articles 10 such as, for example, sanitary napkins, wherein at least one absorbent article 10 can have an enclosed pouch 3 attached thereto. The absorbent articles 10 may all be identical to one another or may be different absorbent articles 10. The package 9 illustrated can be flexible and made from a layer 25. Layer 25 has an interior surface and an exterior surface. The layer 25 may be composed of different materials or may be composed of substantially the same type of material. The layer 25 may be composed of one layer 25 or a laminate. The material can comprise blown or cast film in a blend of low density polyethylene and linear low density polyethylene, metallocenes, ethylene vinyl acetate, surlyn, polyethylene terephthalate, biaxially oriented polypropylene, nylon, or any combination of the aforementioned materials and the like or any other suitable material known to those having ordinary skill in the art. Layer 25 can be folded and secured together to form the package 9. The layer 25 can be secured together by any suitable means discussed herein or any means known in the art. The package 9 can be any shape known in the art such as, for example, a polyhedral shape defining or forming a polyhedral enclosure. While one embodiment can be a flexible polymeric film package is it to be understood that inflexible packages such as those made from cardboard, paperboard, chipboard, plywood, SBS, metal, plastic, paper, card stock, fabric, ceramic, polymer, natural or synthetic fibers, webs, mesh, screen, wood, composite, mixtures, or combinations thereof and the like are also contemplated by the present invention.

The absorbent articles 10 can be arranged to form a stack within the interior space 20 of the package 9. The articles 10 may be stacked in any direction. As used herein, the term “stack” means an orderly pile. For example, the absorbent articles 10 may be stacked vertically, horizontally, or at any angle inside the interior storage space 20 of the package 9.

The package 9 can include a means for accessing the interior storage space 20 for easy access to the absorbent articles 20. The mechanism or means for accessing the interior storage space 20 may include a line of perforations, tabs, adhesive openings or any combination therof and the like or any suitable means for accessing the interior storage space 20 known to those having ordinary skill in the art.

The package 9 further can include at least one region of transparency 12, such as shown in FIG. 7, disposed on at least one package side. As used herein the term “transparent” means having the property of transmitting light without substantial scattering so that bodies lying beyond can be seen relatively clearly by a person having 20/20 vision from a distance of about 12 inches. Positioned adjacent the region of transparency 12 can be an enclosed pouch 3 joined to an absorbent article 10 such that at least a portion of the enclosed pouch 3 and optionally at least some of the indicia 14a and/or 14c contained thereon can be detected by the human eye through the region of transparency 12. It is to be understood that embodiments describing a package 9 having one or more regions of transparency 12 contained on one or more package walls wherein at least one enclosed pouch 3 is positioned adjacent at least one of the one or more regions of transparency 12 such that at least a portion of the enclosed pouch 3 and at least some of the indicia 14a and/or 14c contained thereon are detectable by the human eye through the at least one of the one or more regions of transparency are also contemplated by the present invention.

In additional to communicating to a consumer that there is an absorbent article 10 and an enclosed pouch 3 having a wipe article disposed therein being marketed together through the use of, for example, the region of transparency 12 described hereinabove, it may also be desirable to have a package comprising one or more first articles and one or more second articles each article having indicia that can communicate to the consumer the kind of goods comprising the first and second articles. In addition to communicating the kind of goods comprising the articles it may also be desirable to communicate to a consumer that the goods being marketed together in a common package are the same goods the consumer would purchase individually. It may be even more desirable to communicate to a consumer both of the aforementioned messages simultaneously.

Referring now to FIG. 8 there is shown a package 109 comprising an internal storage space 120, one or more first articles 110 comprising absorbent articles such as, for example, sanitary napkins, one or more second articles 103 comprising enclosed pouches wherein each second article 103 can have a wipe disposed therein. The first article 110 and second article 103 can be joined. The package 109 can also include indicia 111.

The package 109 may include a depiction of a wipe article 111b having a water drop 111a disposed thereon that can communicate to a consumer that there are one or more wet wipes contained in the package 109. The package 109 may still further include indicia 111 such as, for example, a depiction of one or more water droplets 111c, that can relate to the indicia 114 on the one or more second articles 103 viewable by a consumer through one or more regions of transparency 112 as described hereinabove.

In order to communicate to a consumer that the personal care products contained within the package 109 are the same as the products the consumer would normally purchase as separately marketed items, the package 109 may include indicia 111 that can communicate such information to the consumer. For example, if wet wipes are separately marketed in a package having indicia depicting water droplets, and the water droplets are related to, similar to, substantially the same as, or identical to the water droplets depicted on the package containing an absorbent product combination such as the one described hereinabove, then the consumer may feel more comfortable in her purchase knowing that the separately marketed wipes and the wipes in the absorbent product combination are the same wipes.

FIG. 9 shows an example of an alternative embodiment of the present invention. The package 109 of FIG. 9 can comprise an internal storage space 120, one or more first articles 110, and one or more second articles 103. The first article 110 can comprise an absorbent article such as, for example, a sanitary napkin. The second article 103 can comprise an enclosed pouch having a wipe article disposed therein. The first article 110 and second article 103 can be joined. The package 109 further can also include indicia 111.

The package 109 can include a region of transparency 112 that forms a continuous window on two adjacent walls. The region of transparency 112 may comprise a shape that can invite the human eye to investigate the region of transparency 112 such as, for example, the tapered shape shown. In this case a consumer seeing the tapered shape of the window may be more inclined to pick up the package 109 and look at the enclosed pouch 103 and its respective indicia 114a, 114c. This additional interaction by the consumer with the package 109 may result in a higher likelihood of purchase due to the fact that the consumer now has an increased awareness of the increased benefit of the absorbent product combination contained within the package 109.

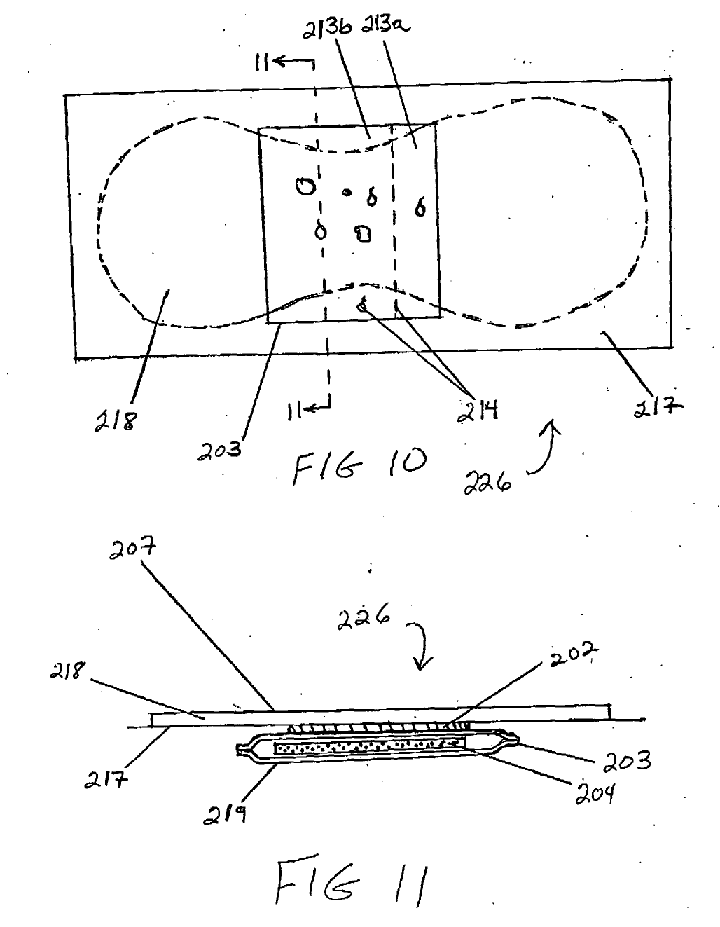

FIG. 10 shows a plan view of another embodiment of the present invention. FIG. 11 shows a cross-section side view of the embodiment in FIG. 10 along line 11. The absorbent product combination 226 shown comprises an absorbent article 218 such as, for example, a panty liner, an enclosed pouch 203 having a wipe 204 disposed therein, a body facing side 207, a garment facing side 208, a joining means 202 for joining the enclosed pouch 203 to the absorbent article 226, and a releasably attached release paper 217.

The release paper 217 shown comprises a rectangular shape, but it is to be understood that other shapes including but not limited to shapes that are similar, substantially the same, or identical to the shape of the absorbent article are contemplated by the present invention. Additionally, the present example shows a release paper 217 that has a greater area than the absorbent article but it is to be understood that release wrappers with an area equal to or less than the area of the absorbent article are also contemplated by the present invention.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A package for storing and dispensing absorbent articles and wipe articles, the package having one or more walls defining an interior storage space and a means for accessing the interior storage space, the package comprising:
a. one or more first articles disposed in the interior storage space wherein at least one of the one or more first articles comprises an absorbent article;
b. one or more second articles having indicia thereon, the second articles disposed in the interior storage space wherein at least one of the one or more second articles comprises an enclosed pouch having a wipe article disposed therein, and wherein at least one of the one or more second articles is joined to at least one of the one or more first articles;
c. one or more sensory cues on the package, wherein the one or more sensory cues on the package relate to the indicia on the one or more second articles; and
d. one or more sensory cues on the package that relate to the wipe article.

2. The package of claim 1 wherein the sensory cue related to the wipe article comprises indicia depicting one or more water droplets.

3. The package of claim 1 wherein the sensory cue related to the wipe article comprises indicia depicting one or more wipe articles.

4. The package of claim 1 wherein the sensory cue related to the wipe article comprises indicia depicting one or more water droplets and one or more wipe articles.

5. The package of claim 1 wherein the indicia on the one or more second packages depict one or more water droplets.

6. The package of claim 1 wherein the indicia on the second article and the indicia on the package are substantially the same.

7. The package of claim 1 wherein the indicia on the second article and the indicia on the package are identical.

8. The package of claim 1 wherein the one or more first articles comprise indicia thereon.

9. An absorbent product combination comprising:
a. a wearable absorbent article;
b. an enclosed pouch having a wipe article disposed therein, the enclosed pouch being joined to the absorbent article by a joining means wherein the joining means has an adhesive force at least as great as the weight of the absorbent product combination, the enclosed pouch having a grasping means comprising:
i. a first grasping portion wherein the first grasping portion comprises about 1%-50% of the surface area of the enclosed pouch and where the first grasping portion enables a user to open the enclosed pouch in order to access the wipe article disposed within, and wherein the force required to open the enclosed pouch is less than the adhesive force of the joining means;
ii. a second grasping portion positioned adjacent to the first grasping portion; and
iii. a sensory cue to communicate to a user the portion of the enclosed pouch that comprises the first grasping portion and the portion of the enclosed pouch that comprises the second grasping portion.

10. A sanitary napkin having a body-facing side, a garment-facing side, a length, a width and two longitudinal side margins, the sanitary napkin comprising:
a. liquid pervious topsheet;
b. a liquid impervious backsheet joined to the topsheet, the backsheet having opposed inwardly and outwardly oriented faces;
c. an absorbent core positioned between the topsheet and the backsheet;
d. a releasable wrapper having opposed outwardly and inwardly facing sides wherein the inwardly facing side is releasably joined to the outwardly oriented face of the backsheet, the releasable wrapper having a longitudinal dimension, a transverse dimension, and inwardly oriented face, an outwardly oriented face, a pair of longitudinal side edges, a pair of end edges, and a pair of ends, wherein the sanitary napkin and the releasable wrapper are folded about two transverse axes which form the wrapper into first, second, and third trisections;
e. an enclosed pouch having a wipe article disposed therein, the enclosed pouch being joined to the sanitary napkin by a joining means, the joining means having an adhesive force at least as great as the weight of the sanitary napkin, the enclosed pouch further comprising an opening means, the opening means comprising:
i. a first grasping portion wherein the first grasping portion comprises about 1%-50% of the surface area of the enclosed pouch and wherein the first grasping portion is at least partially removable such that when the first grasping portion is at least partially removed by a user, the enclosed pouch is opened and the user can access the wipe article contained within the enclosed pouch, and further where the force required to at least partially remove the first grasping portion is less than the adhesive force of the joining means;
ii. a second grasping portion positioned adjacent to the first grasping portion; and
iii. a sensory cue to communicate to a user the portion of the enclosed pouch that comprises the first grasping portion and the portion of the enclosed pouch that comprises the second grasping portion.

11. The sanitary napkin of claim 10 wherein the force required to at least partially remove the first grasping portion is equal to or greater than the adhesive force of the joining means.

12. The sanitary napkin of claim 10 wherein the force required to at least partially remove the first grasping portion is about 0.5 N to about 15 N.

13. The sanitary napkin of claim 10 wherein the force required to at least partially remove the first grasping portion is about 1 N to about 10 N.

14. The sanitary napkin of claim 10 wherein the force required to at least partially remove the first grasping portion is about 6 N.
